(19) United States
(12) Patent Application Publication
(10) Pub. No.: US 2024/0261450 A1
(43) Pub. Date: Aug. 8, 2024

(54) DEVICE AND METHOD FOR DISINFECTING A WORKING VOLUME IN AN ARTIFICIAL SPACE ENVIRONMENT VIA SOLAR UV RADIATION

(71) Applicant: ISTITUTO NAZIONALE DI ASTROFISICA, Roma (IT)

(72) Inventors: Primo ATTINA, Roma (IT); Andrea BIANCO, Roma (IT); Fausto CORTECCHIA, Roma (IT); Emiliano DIOLAITI, Roma (IT); Luigi LESSIO, Roma (IT); Matteo LOMBINI, Roma (IT); Alberto MACCHI, Roma (IT); Giuseppe MALAGUTI, Roma (IT); Giovanni PARESCHI, Roma (IT); Maria Guglielmina PELIZZO, Roma (IT)

(21) Appl. No.: 18/566,210

(22) PCT Filed: Jun. 3, 2022
(86) PCT No.: PCT/IB2022/055198

§ 371 Date: Dec. 1, 2023

(30) Foreign Application Priority Data
Jun. 4, 2021 (IT) 102021000014657

(51) Int. Cl. A61L 2/10; A61L 2/26; A61L 9/20; B64G 1/48; F21S 11/00; F21V 9/04

(52) U.S. Cl. CPC A61L 2/10; A61L 2/26; A61L 9/20; B64G 1/48; F21S 11/007; F21V 9/04; G02B 6/0006; A61L 2202/11; A61L 2202/25; A61L 2209/12

(57) ABSTRACT

Device for disinfecting at least one working volume in an artificial space environment, comprising a concentrator of solar radiation associated with the at least one working volume, and filtering means for selecting at least one spectral band of the solar radiation comprising UVC radiation coupled to the concentrator.

### CROSS-REFERENCE TO RELATED APPLICATIONS

This patent application claims priority of Italian Patent Application No. 102021000014657 filed on Jun. 4, 2021, the entire disclosure of which is incorporated herein by reference.

### TECHNICAL FIELD

The present invention relates to a device and method for disinfecting a working volume in an artificial space environment via solar UV radiation.

### BACKGROUND ART

As is known, the Sun emits radiation in all the bands of the electromagnetic spectrum, with varying intensity depending on the wavelength. In particular, the intensity is greater in the intermediate region of the electromagnetic spectrum, which comprises ultraviolet (UV), visible (Vis) and infrared (IR) radiations, respectively in ascending order of wavelength (and thus in descending order of frequency).

UV radiation, also known as UV rays, comprises the wavelength range from 200 nm to 400 nm. Conventionally, three UV ray ranges are considered when analysing the effects on human health:
UVA (so-called long-wave UV), from 400 nm to 315 nm, useful for stimulating vitamin D production in the human body, and weakly absorbed by DNA and RNA due to their relatively low frequency;
UVB (so-called medium-wave UV), from 315 nm to 280 nm; and
UVC (so-called short-wave UV), from 200 nm to 250 nm, whose high frequency is capable of modifying the DNA or RNA of microorganisms, enabling a germicidal action.

The visible spectrum comprises the wavelength range from about 400 nm (red colour) to 700 nm (violet colour), including all colours perceptible to the human eye. Therefore, radiation in this range of the electromagnetic spectrum is called light.

IR radiation, also known as IR rays, comprises the wavelength range from 700 nm to 1 mm. There are several conventions for classifying IR rays. In some of them, the range of IR radiation bordering the visible spectrum is called NIR (“near-infrared”), and is followed (starting from about 3500 nm) by MIR (“mid-infrared”) or thermal infrared.

When crossing the Earth's atmosphere, solar radiation undergoes phenomena of reflection, refraction, absorption and diffusion, by the various atmospheric gases and to a varying extent depending on the wavelength. Therefore, the solar spectrum measured on the Earth's surface is different from that detected at the outer thresholds of the Earth's atmosphere.

In particular, the Earth's atmosphere absorbs almost all of UVC and a high percentage of UVB, thus most of the UV rays reaching the Earth's surface are UVA. Therefore, for terrestrial applications, UVC radiation is only available from artificial sources. For this purpose, germicidal lamps are known and used to produce UVC radiation to kill and/or inactivate microorganisms (including fungi and moulds).

The adoption of disinfection techniques presents even greater problems in artificial space environments, e.g. space stations, spacecraft, and lunar and planetary bases.

The purpose of the present invention is to realise a device for disinfecting that can be used in an artificial space environment.

### DISCLOSURE OF INVENTION

The aforementioned purpose is achieved by a device for disinfecting as claimed in claim 1.

The present invention also relates to a method for disinfecting as claimed in claim 15.

### BRIEF DESCRIPTION OF DRAWINGS

For a better understanding of the present invention, a preferred embodiment is described below, by way of non-limiting example and with reference to the accompanying drawings, wherein:

FIG. 1 is a schematic perspective view of an artificial space environment comprising a device according to the present invention;

FIG. 2 is a schematic front view of a detail of FIG. 1;

FIGS. 3a, 3b are axial sectional views of a component of the device of FIG. 1;

FIG. 4 is a perspective view of a variant of the component of FIG. 3;

FIGS. 5, 6 are sectional views of further variants of the component of FIG. 3;

FIGS. 7a, 7b, 8 are axial sectional views of components of the device of FIG. 1, according to different variants;

FIGS. 9a, 9b, 10 are schematic front views of variants of the device of FIG. 1;

FIG. 11 is a sectional view of a component of the device of FIG. 10; and

FIG. 12 is a block diagram of the control of the device of FIG. 1.

### DETAILED DESCRIPTION OF THE INVENTION

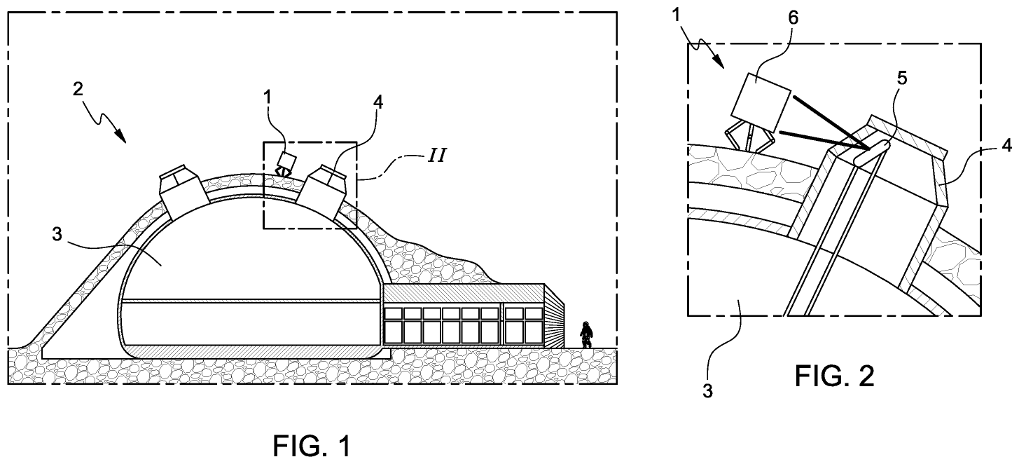

With reference to FIG. 1, there is indicated by 1 a device for disinfecting according to the present invention. The device 1 is arranged externally to an artificial space environment 2. The artificial space environment 2 can be e.g. a space station, a spacecraft, a lunar base, or a planetary base (e.g. on Mars). By way of example, FIG. 1 shows a schematic diagram of a lunar base 2 comprising a main environment 3, at least partially protected by compressed regolith, from which one or more domes 4 extend upwards emerging from the cover to allow observation and natural illumination. The dome 4 houses a portion of a conditioning system comprising a tank defining a working volume 5, a first conduit at the inlet of the tank for the supply of fluid to be sterilised, e.g. air coming from previous stages of a regeneration plant, and a second conduit at the outlet of the tank for the re-injection of the sterilised fluid, e.g. air re-injected into the main environment 3 or into the regeneration plant. The device 1 faces the tank through a window which is transparent to radiation of a predetermined wavelength, as described in detail hereinafter.

It should be noted that the term “working volume” is used here in its broadest sense and can comprise any closed or open volume, such as a conduit, a tank, a habitable room.

The device 1 comprises a concentrator 6 of solar radiation associated with the working volume 5, and a filter 7 coupled to the concentrator 6.

Optionally, the device 1 may comprise an optical transport means 8 of the radiation from the concentrator 6 to the at least one working volume 5 and/or a system 9 for moving the concentrator 6.

The concentrator 6 can be static or dynamic.

The static concentrators 6 are designed to receive solar radiation without requiring any movement.

The dynamic concentrators 6 are used if the direction of incidence of the solar radiation has a range of variability greater than the angle of acceptance of the concentrator 6, thus they need the system 9 for moving the concentrator 6 to be able to collect solar radiation effectively, as described below. Speed and accuracy of the movement system 9 depend mainly on where the concentrator 6 is placed. For example, at the poles of the Moon, the Sun is always visible and the direction of arrival of the solar radiation varies with the Moon's period of revolution around the Earth (about 27 days), whereas on a travelling spacecraft (e.g. to Mars) or an orbiting space station, the variation of the Sun's position with respect to concentrator 6 might vary differently.

The choice of the concentrator 6 depends on the type of application.

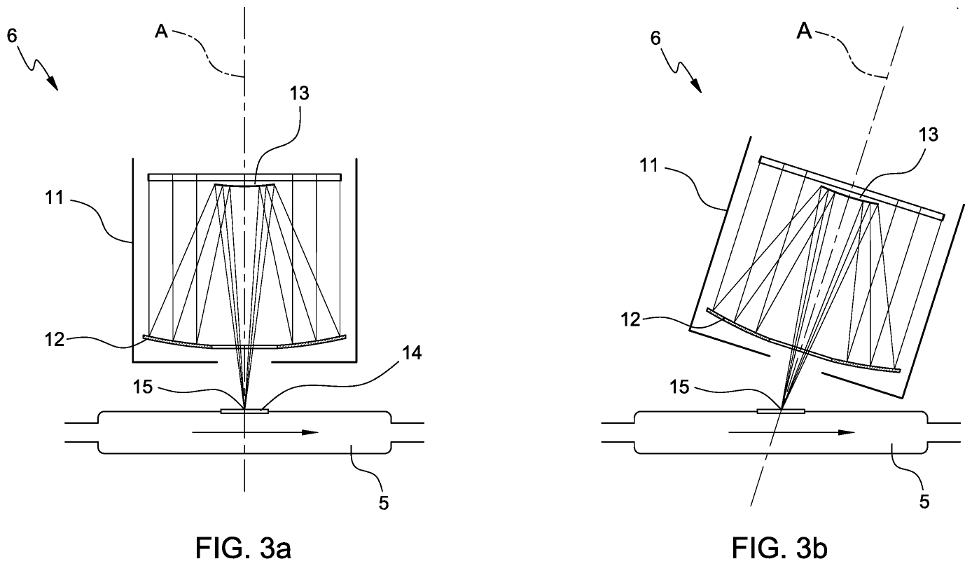

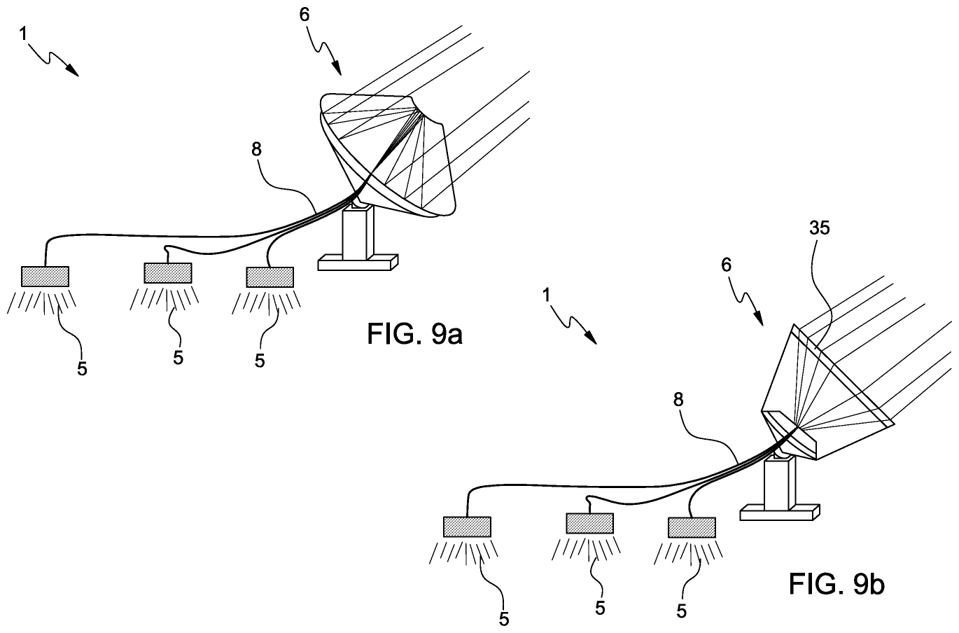

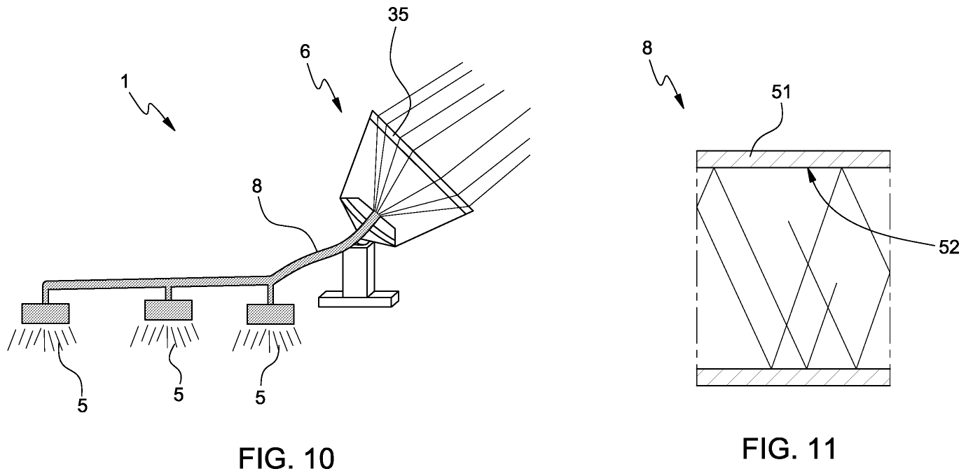

In a first embodiment (FIGS. 3a, 3b), the concentrator 6 is a mirror system, e.g. in a Ritchey-Chrétien optical configuration, comprising an A-axis cylindrical casing 11, a primary mirror 12 and a secondary mirror 13, e.g. hyperbolic, which are arranged within the cylindrical casing 11. The cylindrical mirror 12, of axis A, reflects the solar rays (which can be considered parallel to one another) concentrating them towards the secondary mirror 13, coaxial to the primary mirror 12 and facing it, which reflects them concentrating them in a focal plane 14, orthogonal to the axis A and passing through a point 15 of the axis A called telescope focus. The focal plane 14 can be directly inside (FIGS. 3a, 3b) the working volume 5, or the solar radiation can be transported from the focal plane 14 to the working volume 5 (FIGS. 9a, 9b, 10) via the optical transport means 8, described below. For an optimal use of this concentrator 6, the axis A must be oriented parallel to the solar rays. Therefore, this concentrator 6 is preferably dynamic, thus being able to be moved so as to assume different orientations to follow the apparent position of the Sun (FIG. 3b).

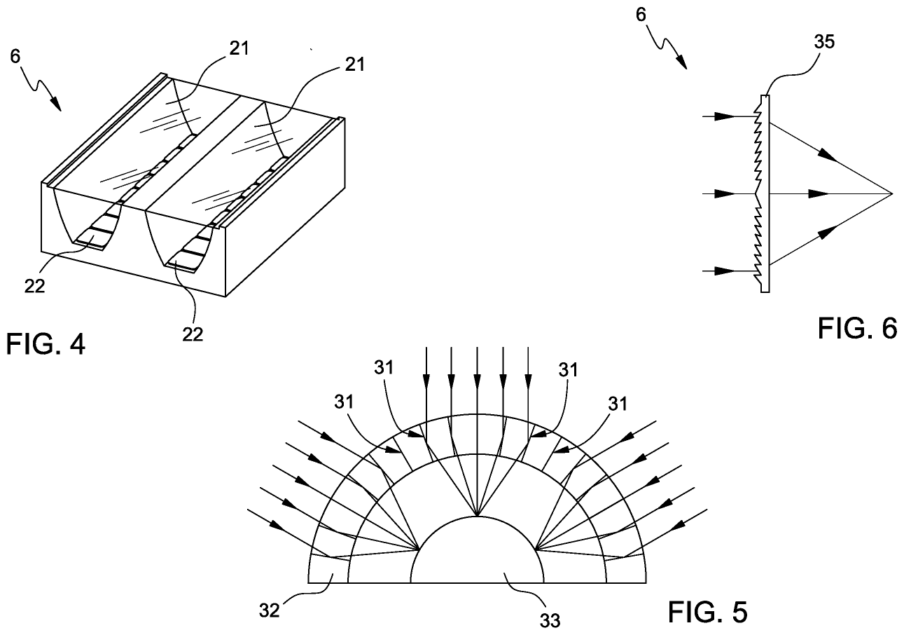

In a second embodiment (FIG. 4), the concentrator 6, of parabolic type, comprises at least one mirror 21 having the shape of a parabolic cylinder, optionally truncated at a focal plane 22 thereof, which reflects and concentrates the solar rays on the focal plane 22. This concentrator 6 can have an angle of acceptance of some tens of degrees, and can thus be static.

In a third embodiment (FIG. 5), the concentrator 6, of “lobster eye” type, comprises a series of flat mirrors 31, extending along radii of a spherical half-crown 32, which reflect the solar rays, irrespective of their direction, towards a semispherical detector 33. In particular, incident rays parallel to one another are reflected in a same point on the detector 33. This concentrator 6 allows to concentrate the solar rays coming from any direction of a hemisphere, and can thus be static.

According to another embodiment (FIG. 6), instead of a reflection concentrator 6 as those described above, a transmission concentrator 6 may be used, for example comprising a Fresnel lens 35.

The filter 7 allows the selection of desired spectral bands and the rejection of unwanted spectral components, e.g. because they are harmful.

The choice of the desired spectral band depends on the type of application.

For example, UVC radiation, naturally present in the extra-terrestrial environment, can be used to sterilise the artificial space environment 2, as its germicidal action enables the sanitization of air, water or surfaces.

UVB radiation, due to its greater wavelength with respect to UVC radiation, is less efficient as a germicide, thus not being used for this purpose on Earth. However, UVB radiation could be used as a germicide in the extra-terrestrial environment, since the integrated emission (power emitted per unit area) of the solar radiation in space is much higher than that on the Earth's surface, due to the filtering action of the Earth's atmosphere.

UVA radiation could be used for hydroponic cultures and/or to stimulate vitamin D production in the human body.

The visible spectrum could be used for illuminating the environment, e.g. the main environment 3.

Depending on the filter 7, it is possible to select either a narrow spectral band, e.g. comprising UVC radiation alone or UVC and UVB radiation for their germicidal action, or a broader spectral band, e.g. comprising the entire UV radiation and the visible spectrum, from which the various components can be separated later for the different applications (disinfection, hydroponics, illumination), e.g. via dichroic filters.

In general, the rejection of the harmful spectral components of solar radiation is desirable.

For example, in the UV ray band with wavelengths shorter than UVC, in particular at 185 nm, ozone, which is toxic, is formed; therefore, it is advisable to cut the spectral band lower than 220 nm.

On the other hand, considering wavelengths greater than NIR, another potentially harmful spectral band is MIR or thermal infrared, as it could cause overheating and degradation of materials or even fires, when the irradiation per unit area exceeds certain values.

In the following, the filter 7 coupled to the concentrator 6 described above in the first embodiment is described.

The filter 7 allows a transmission and/or reflection filtering.

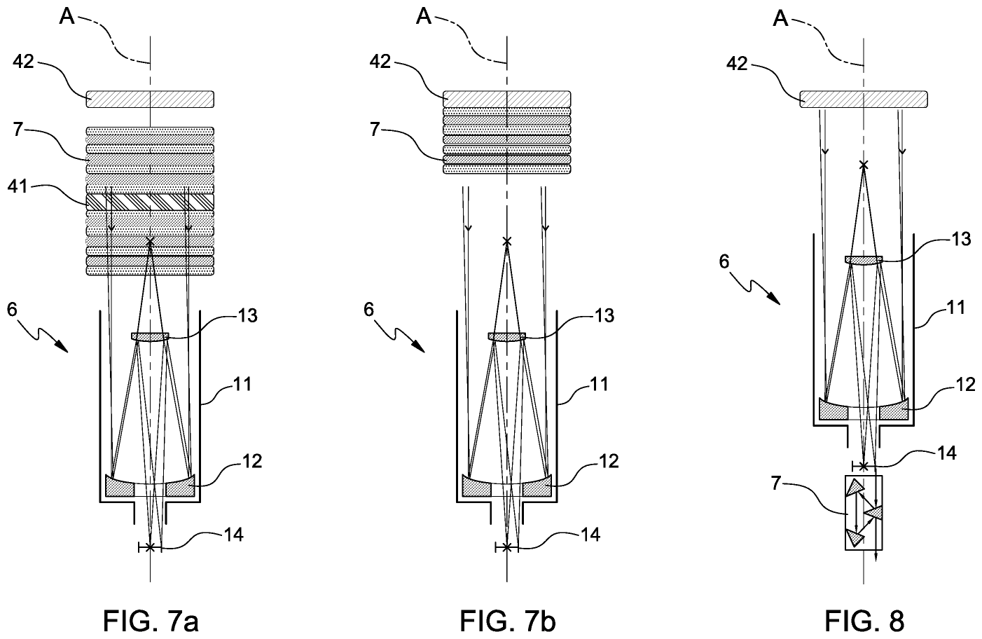

In transmission filtering (FIGS. 7a, 7b), the filter 7 is a bandpass interference filter arranged coaxially to the concentrator 6 and upstream of it, i.e. on the side opposite to the focal plane 14. Depending on the filter 7, it is possible to select the desired band, e.g. UVC, and eliminate the other bands. In order to increase rejection in the NIR band, a substrate 41, preferably made of glass with UG equal to 5, can be added to the filter 7. In order to abate the MIR band, it is possible to use a quartz window 42, parallel to the filter 7 which is interposed between it and the concentrator 6. In particular, the filter 7 can be spaced from the window 42 (FIG. 7a), or deposited directly on it (FIG. 7b).

In reflection filtering (FIG. 8), the filter 7 is a multiple reflection filter system, extending substantially along the axis A, downstream of the concentrator 6. Similarly to the transmission filtering, a quartz window 42, extending orthogonally to the axis A, can be used to abate the MIR band.

In a variant (not shown), reflection filtering can be realised by depositing respective reflection filters on the primary mirror 12 and on the secondary mirror 13 of the concentrator 6.

In transmission and reflection filtering, the filter 7 comprises a substrate of high-pass material in transmission, arranged upstream of the concentrator 6, and a low-pass interference filter in reflection, arranged downstream of the concentrator 6. Therefore, a bandpass filter 7 is realised through a mixed system. Similarly to the previous cases, a quartz window 42, parallel to the transmission filter that is interposed between it and the concentrator 6, can be used to abate the MIR band.

The following are the results of simulations performed with reference to a Ritchey-Chrétien type mirror system as shown in FIGS. 3a, 3b, wherein the primary mirror has a diameter of 0.50 m with a central obstruction equal to one third of the diameter, the air inlet conduit has a diameter of 0.14 m, the air inlet speed is equal to 6.5 m/s and the working volume consists of a conduit with a diameter of 0.266 m and a length equal to 1 m. An integrated UVC radiation flux in the 220-280 nm band equal to 0.6 mW/cm² and an overall efficiency of the mirror and filter system equal to 0.6 is assumed, resulting in a UVC flux equal to about 0.6 W.

Assuming that the walls of the working volume have a reflectance equal to 0.95 and Lambertian scattering, a UVC radiation dose on each air element crossing the working volume equal to 15 mJ/cm² is obtained.

Using a mirror system with a double-diameter primary mirror would result in a fourfold flux and dose (equal to about 2.5 W and 60 mJ/cm², respectively).

For comparison, the average 90% inactivation of viruses and bacteria occurs with 0.6 mJ/cm², of bacterial spores with 9 mJ/cm², of fungi with 2.3 mJ/cm², of fungal spores with 31 mJ/cm² (source: Ultraviolet Germicidal Irradiation Handbook, W. Kowalsky, Springer 2009, Table 4.1).

UV radiation with a wavelength greater than 280 nm is not considered here, which however contributes, albeit with a minor effect, to the germicidal action.

If present, the optical transport means 8 (FIGS. 9a, 9b, 10) of the radiation allows to connect the concentrator 6 to the at least one working volume 5, in case they are distant from each other.

In FIG. 9a the concentrator 6 is of the reflection type, in particular a telescope, in FIGS. 9b and 10 it is of the transmission type, in particular a Fresnel lens 35.

The optical transport means 8 may comprise optical fibres (FIGS. 9a, 9b), preferably with low absorption in the spectral bands of interest, or reflective conduits 51 (FIG. 10). In particular, the reflective conduits 51 (FIG. 11), preferably cylindrical, comprise internal walls 52 coated with reflective material that allow radiation to propagate by subsequent reflections. Since the characteristics of the reflective material of the internal walls 52 affect the reflection efficiency of the reflective conduits 51, it is possible to choose a coating optimised for reflecting radiation in the spectral bands of interest, so as to transport radiation over relatively long distances with low losses.

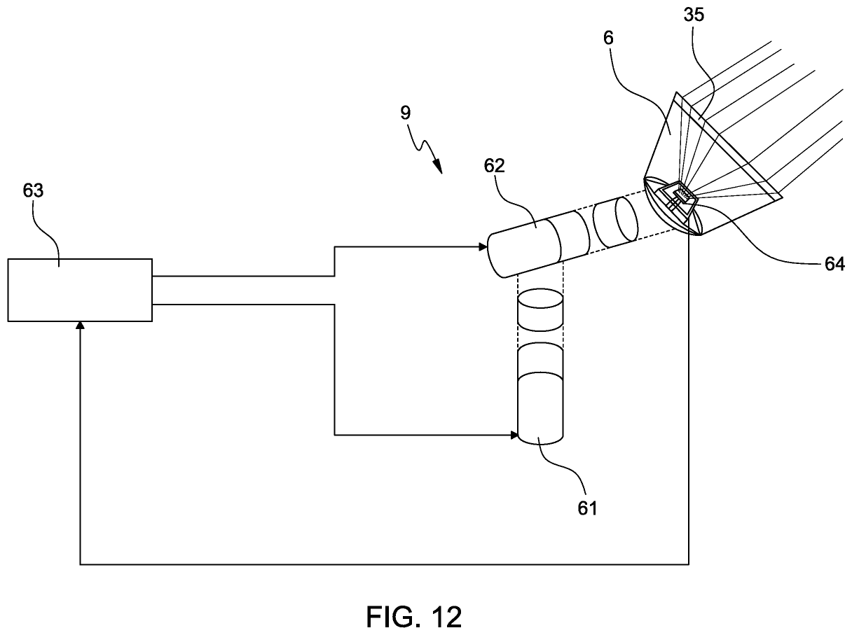

If present, the movement system 9 (FIG. 12) allows to obtain a dynamic concentrator 6, which can assume a plurality of different configurations to collect solar radiation effectively.

In the following, the system 9 for moving the concentrator 6 with Fresnel lens 35 is described.

The movement system 9 allows the Sun to be followed so as to have the solar radiation always centred on the focal plane 14 of the concentrator 6.

The movement system 9 can be realised in different ways.

For example, the movement system 9 can allow an azimuth movement and a height movement, controlled by respective motors 61, 62 via a closed-loop control realised by a microprocessor 63, by a four-quadrant sensor 64 with centre on the axis A and by an electronics that measures, instant by instant, the signal difference existing between the quadrants of the sensor 64. Therefore, the control tends to align the axis A with the direction of maximum radiation.

Upon examination of the characteristics of the device 1, the advantages of the present invention are clear.

In particular, it is possible to exploit spectral bands useful for sterilisation, in particular the UVC band that is not available on Earth.

Optionally, different spectral bands may be exploited for different purposes.

If present, the optical transport means 8 of the radiation allows greater flexibility in placing the concentrator 6 and/or the at least one working volume 5.

Finally, it is clear that modifications and variations can be made to the device 1 without going beyond the scope of protection defined by the claims.

1. Device for disinfecting at least one working volume in an artificial space environment, comprising a concentrator of solar radiation associated with the at least one working volume, and filtering means for selecting at least one spectral band of the solar radiation comprising UVC radiation coupled to the concentrator.

2. Device as claimed in claim 1, comprising optical transport means of the filtered solar radiation to connect the concentrator to the at least one working volume.

3. Device as claimed in claim 2, wherein the optical transport means comprise optical fibers.

4. Device as claimed in claim 2, wherein the optical transport means comprise reflective conduits.

5. Device as claimed in claim 1, comprising means for moving the concentrator to maintain a direction of incidence of the solar radiation within an angle of acceptance of the concentrator.

6. Device as claimed in claim 1, wherein the concentrator is a mirror system.

7. Device as claimed in claim 6, wherein the mirror system is in a Ritchey-Chrétien optical configuration.

8. Device as claimed in claim 1, wherein the concentrator is selected from the group comprising a parabolic type concentrator, a “lobster eye” type concentrator, and a concentrator comprising a Fresnel lens.

9. Device as claimed in claim 1, wherein the spectral band of the solar radiation comprises UVB radiation.

10. Device as claimed in claim 1, wherein the filtering means comprise a transmission filter.

11. Device as claimed in claim 1, wherein the filtering means comprise a reflection filter.

12. Device as claimed in claim 1, wherein the filtering means comprise a filter for rejection of an infrared band.

13. Device as claimed in claim 12, wherein the filter comprises a quartz window.

14. Artificial space environment comprising at least one working volume and a device as claimed in claim 1.

15. Method for disinfecting at least one working volume in an artificial space environment, comprising the steps of concentrating solar radiation towards the at least one working volume and filtering the solar radiation to select at least one spectral band of the solar radiation comprising UVC radiation.

16. Method as claimed in claim 15, comprising the step of transporting the filtered solar radiation towards the at least one working volume.

17. Method as claimed in claim 15, wherein the spectral band of the solar radiation comprises UVB radiation.
